(19) United States
(12) Patent Application Publication
(10) Pub. No.: US 2014/0163044 A1
(43) Pub. Date: Jun. 12, 2014

(54) Compound Chemical Medicine Acting on Respiratory Disease, Preparation Process and Use Thereof

(71) Applicants: Zhihong Zhu, Changsha (CN); Zhenjun Bo, Changsha (CN); Jing Yang, Changsha (CN)

(72) Inventors: Zhihong Zhu, Changsha (CN); Zhenjun Bo, Changsha (CN); Jing Yang, Changsha (CN)

(73) Assignee: HUNAN JIUDIAN PHARMACEUTICAL CO., LTD., Hunan (CN)

(21) Appl. No.: 14/122,875

(22) PCT Filed: May 29, 2012
(86) PCT No.: PCT/CN2012/076191

§ 371 Date: Dec. 10, 2013

(30) Foreign Application Priority Data
Jun. 2, 2011 (CN) 201110146717.3

(51) Int. Cl. A61K 31/495; A61K 31/197

(52) U.S. Cl. CPC A61K 31/495; A61K 31/197
USPC 514/255.03

(57) ABSTRACT

A pharmaceutical composition for respiratory system disease and the preparation process therefor, consisting of levodropropizine and carbocisteine as active ingredients with a pharmaceutically acceptable excipient. The present pharmaceutical composition has a marked cough-relieving effect and fewer negative reactions.

This application claims the priority of China Patent Application No. 201110146717.3, filed with the Patent Office of China on Jun. 2, 2011, titled “COMPOUND CHEMICAL MEDICINE ACTING ON RESPIRATORY DISEASE, PREPARATION PROCESS AND USE THEREOF”, the contents of which are incorporated herein by reference in its entirety.

### FIELD OF THE INVENTION

The present invention relates to the field of chemical medicine, to an antitussive and expectorant pharmaceutical composition as well as the preparation process and use thereof, particularly to a pharmaceutical composition comprising levodropropizine and carbocisteine as active ingredients.

### BACKGROUND OF THE INVENTION

With the development of modern industry, the air pollution is increasingly serious, and the incidence of respiratory system disease is rising, the main clinical symptoms thereof are cough, sputum and panting. Therefore, treatment of respiratory system disease also requires etiological treatment and symptomatic treatment. If the phlegm or foreign body can be expelled, mild and infrequent cough can spontaneously remit without medication. However, too frequent severe cough, if left untreated, will seriously affect a person's life, work and rest, not only increasing the person's agony, affecting rest and sleep, increasing physical consumption, and even likely inducing the onset and development of other latent disease such as pneumonia, chronic pharyngitis, chronic bronchitis, bronchiectasis, lung abscess and cavitary pulmonary tuberculosis. At this time, in addition to the right medicine for symptomatic treatment against respiratory system diseases, appropriately applying antitussive and expectorant medicines is also needed to relieve cough.

Currently, the antitussives commonly used are central antitussives, such as codeine, dextromethorphan and pentoxyverine, and expectorants include ammonium chloride, guaifenesin, etc. Wherein, dextromethorphan hydrobromide, which is a medicine similar to the narcotic analgesic levorphanol, inhibits the cough center in the spinal cord or related higher nerve center to suppress the cough reflex, i.e. this medicine directly acts on the central nervous system. Although there is currently no report related to the addiction or drug resistance of this medicine, it has many side effects, and is likely to induce nausea, vomiting, constipation, drug resistance to antitussive and analgesic effects, as well as addiction. In May 2005, the FDA announced that 5 children in the US died from excessively taking capsules containing “dextromethorphan”. Then, the State Food and Drug Administration of China released alert on not taking an overdose of dextromethorphan. Since then, the compound preparation containing dextromethorphan faces a dilemma. Clinical trials demonstrate that an ideal antitussive should reduce the sensitivity of pulmonary sensory nerves, block sensory nerve conduction in myelinated or unmyelinated nerves (such as a local anesthetic), but local anesthetics have side effects of inhibiting pulmonary defense reflex, inducing bronchoconstriction, etc.

Levodropropizine is a chiral peripheral antitussive, first developed by Italy's Dompe company (trade name levotuss) and Mediolanum company (trade name Danka), and it was marketed in Italy in October 1988. Studies have confirmed that levodropropizine can significantly reduce the response of the vagal afferent C-fibres to chemical stimuli, and have a peripheral mechanism of action, partially inhibit the effect of histamine and the effect of promoting the release of neuropeptide. Due to the advantages that levodropropizine has good selectivity, strong antitussive effect, and almost no effect on the central nervous system, no side effects such as drowsiness, respiratory depression, dependence, etc., and produces no significant effects on the cardiovascular and respiratory systems, we chose levodropropizine as the antitussive for the pharmaceutical composition of the present invention.

Carbocisteine, which is a phlegm-dissolving agent, has a direct effect on bronchial glands after oral administration, prompts the release of lysosomes by mucus-secreting cells, disintegrates and lyses the sticky sugar fibers in the phlegm. It can also inhibit the synthesis of acidic glycoprotein in mucus glands and goblet cells, so that small molecular glycoprotein with lower viscosity is secreted, which lowers the viscosity of the phlegm, and makes it easy to be coughed out. Therefore, levodropropizine and carbocisteine are combined together as active ingredients of the compound preparation. For a patient with a respiratory system disease, they play antitussive and expectorant effects, meanwhile, have less adverse reactions and higher safety. Therefore, the present invention will bring some gospel to a patient, and has some social and economic significance.

### SUMMARY OF THE INVENTION

The objective of the present invention is to provide an antitussive and expectorant pharmaceutical composition consisting of levodropropizine and carbocisteine, for treating respiratory system disease.

The objective of the present invention is achieved by the following means.

The active pharmaceutical ingredients of the pharmaceutical composition of the present invention are the antitussive medicine levodropropizine and the phlegm-dissolving agent carbocisteine. Both of them are formulated by the weight ratio of 1˜10 parts of levodropropizine to 15˜45 parts of carbocisteine.

The active ingredients levodropropizine and carbocisteine can also be formulated by the weight ratio of 3˜8 parts of levodropropizine to 20˜40 parts of carbocisteine.

The active ingredients levodropropizine and carbocisteine can also be formulated by the weight ratio of 4˜7 parts of levodropropizine to 25˜37.5 parts of carbocisteine.

The active ingredients levodropropizine and carbocisteine are optimally formulated by the following weight ratio: the active ingredients levodropropizine and carbocisteine are formulated by the weight ratio of 6 parts of levodropropizine to 25 parts of carbocisteine; or 6 parts of levodropropizine to 37.5 parts of carbocisteine.

Another objective of the present invention is to provide a method for preparing an antitussive and expectorant pharmaceutical composition. The active ingredients are formulated with any pharmaceutically acceptable excipient into an immediate-release pharmaceutical preparation, a slow-release pharmaceutical preparation or a common pharmaceutical preparation by suitable preparation means of the pharmaceutical preparation.

The pharmaceutical preparation formulated by active ingredients and any pharmaceutically acceptable excipient of the present invention may be any oral preparation in pharmaceutics.

The oral preparation may be a tablet, a capsule or an oral liquid, etc.

The method for preparing the oral tablet of the present invention is as follows:
A. weighing levodropropizine and carbocisteine respectively in accordance with each weight ratio mentioned above;
B. sieving the weighed levodropropizine and carbocisteine respectively, and then mixing them evenly according to the method of increment by equal quantity;
C. weighing appropriate amount of excipients including hydroxypropyl methyl cellulose (k15m) and hydroxypropyl methyl cellulose (k100), magnesium stearate and microcrystalline cellulose;
D. mixing the hydroxypropyl methyl cellulose (k15m) and the hydroxypropylmethyl cellulose (k100) evenly, followed by mixing with microcrystalline cellulose, then adding the mixture obtained in Step B and mixing evenly, sieving and granulating, drying, granule sizing, adding the weighed magnesium stearate, mixing the mixture evenly and compressing into tablets, to obtain tablets of the composition of the present invention.

For the antitussive and expectorant pharmaceutical composition of the present invention, the oral daily dose is 60˜200 mg levodropropizine and 0.5˜1.5 g carbocisteine, 1˜3 times a day.

The pharmacodynamic test for the pharmaceutical composition of the present invention:
1. Studies on the Antitussive Effect

Medicines used in the test:

Test medicine: self-made tablets of the pharmaceutical composition comprising levodropropizine and carbocisteine by the inventors

Positive control medicine: levodropropizine tablets and carbocisteine tablets

Test method:

150 guinea pigs (half male and half female) with a body mass of 300±25 g are taken and evenly divided into 15 groups, with 10 in each group. The administration group for the tablet of the present invention is divided into high dose group, medium dose group and low dose group, and the tablets are orally administered respectively; the administration groups for the positive control medicine levodropropizine is also divided into high dose group, medium dose group and low dose group. Each dose group is divided into two groups, respectively the group inducing cough at the first hour or the fourth hour after administration. Doses in each group and the test results are as shown in the following Table 1˜2:

TABLE 1
Antitussive effect for the composition of the present
invention in guinea pigs 1 hour after administration

| | | Cough times | | |
|---|---|---|---|---|
| | | before | One hour after administration | |
| | Dose | administration | Latent period | Cough times |
| Groups | (mg/kg) | (in 5 min) | of cough (s) | (min) |
| Distilled water | — | 25.05 ± 3.87 | 24.52 ± 8.75 | 25.10 ± 5.01 |
| group | | | | |
| High dose group of | 120 | 25.18 ± 5.31 | 49.57 ± 6.87 | 14.21 ± 4.25 |
| levodropropizine | | | | |
| tablet | | | | |
| Medium dose group of | 60 | 25.41 ± 5.26 | 42.38 ± 5.98 | 17.64 ± 3.97 |
| levodropropizine | | | | |
| tablet | | | | |
| Low dose group of | 30 | 25.14 ± 4.37 | 38.16 ± 5.88 | 19.06 ± 4.15 |
| levodropropizine | | | | |
| tablet | | | | |
| High dose group of the | 120 | 25.31 ± 4.26 | 53.41 ± 5.73 | 10.15 ± 5.17 |
| present invention | | | | |
| Medium dose group of the | 60 | 25.09 ± 4.97 | 47.10 ± 6.31 | 12.25 ± 3.78 |
| present invention | | | | |
| Low dose group of the | 30 | 25.19 ± 4.65 | 44.67 ± 5.97 | 14.84 ± 5.47 |
| present invention | | | | |
| Carbocisteine tablet | 60 | 25.24 ± 4.53 | 31.28 ± 6.42 | 21.15 ± 3.98 |
| group | | | | |

TABLE 2
Antitussive effect for the composition of the present
invention in guinea pigs 4 hour after administration.

| | | Cough times | | |
|---|---|---|---|---|
| | | before | Four hours after administration | |
| | Dose | administration | Latent period | Cough times |
| Groups | (mg/kg) | (in 5 min) | of cough (s) | (min) |
| Distilled water | — | 26.15 ± 4.34 | 25.34 ± 6.49 | 25.83 ± 4.94 |
| group | | | | |
| High dose group of | 120 | 26.27 ± 3.98 | 42.18 ± 3.18 | 15.13 ± 3.97 |
| levodropropizine | | | | |
| tablet | | | | |
| Medium dose group of | 60 | 25.89 ± 4.06 | 40.27 ± 5.29 | 17.21 ± 3.45 |
| levodropropizine | | | | |
| tablet | | | | |
| Low dose group of | 30 | 25.74 ± 3.71 | 36.34 ± 4.68 | 19.41 ± 5.03 |
| levodropropizine | | | | |
| tablet | | | | |
| High dose group of the | 120 | 26.98 ± 3.96 | 50.36 ± 5.38 | 11.85 ± 4.56 |
| present invention | | | | |
| Medium dose group of the | 60 | 26.86 ± 4.61 | 45.27 ± 4.59 | 13.17 ± 3.91 |
| present invention | | | | |
| Low dose group of the | 30 | 25.95 ± 3.87 | 42.01 ± 4.13 | 15.94 ± 4.58 |
| present invention | | | | |
| Carbocisteine tablet | 60 | 26.04 ± 4.37 | 32.56 ± 5.17 | 20.04 ± 4.24 |
| group | | | | |

The above results indicate that the tablet of the present invention and levodropropizine in all the groups have effects of prolonging latent period of cough and reducing cough times during the treatment. In a situation of the same dose, the effect of the present invention tablet is significantly better than that of the levodropropizine tablet. The efficacy of the low dose group of the present invention is equivalent to that of the high dose group of levodropropizine tablet, and the results are statistically significant. While the carbocisteine group has no obviously effect on prolonging the latent period of cough and reducing the cough times during the treatment. From the above test results, it can be known that the tablet of the present invention has better efficacy than that of the levodropropizine tablet or the carbocisteine tablet in prolonging latent period of cough and reducing the cough times during the treatment.
2. Trials for Observing the Clinical Effect

Trials for observing the clinical effect of the tablet of the present invention was conducted in the People's Hospital of Yongshun County, Hunan Province.

300 outpatients or inpatients with cough caused by acute upper respiratory tract infection, acute tracheitis and bronchitis, community-acquired pneumonia, and acute exacerbation of chronic bronchitis were randomly divided into a treatment group and a placebo group, 100 patients for each group. Medicines were administered following the double-blind double-dummy positive medicine parallel control method. The tablets of the present invention, the control medicine levodropropizine tablets or carbocisteine tablets were taken by each group, respectively. Each group orally takes the tablets 3 times a day, one tablet (including analog tablet) each time, for a 5-day course of treatment. Other antitussive or expectorant medicine was stopped during the administration. The patients' conventional indicators such as blood pressure, pulse rate, body temperature, respiration, etc.; symptoms of cough; chest x-ray; blood and urine routine tests; liver and kidney functions; adverse reaction records and electrocardiogram were recorded.

Criteria for evaluating the efficacy are as follows: clinically controlled: symptoms disappeared completely; effective: symptoms of cough reduced to 1 point from 3 points; improved: symptoms of cough reduced to 2 points from 3 points or reduced to 1 points from 2 points; ineffective: symptoms of cough were not improved or aggravated.

The results of the trial are as follows:

The efficacy table for the clinical observation of the tablet of the present invention:

| | | Efficacy (cases) | | | | | |
|---|---|---|---|---|---|---|---|
| | | Clinically | | | | Effective | |
| Medicine | Cases | controlled | Effective | Improved | Ineffective | rate | P |
| The tablet of the | 100 | 69 | 20 | 10 | 1 | 99.0 | <0.05 |
| present invention | | | | | | | |
| Levodropropizine | 100 | 60 | 15 | 17 | 8 | 92.0 | <0.05 |
| tablet | | | | | | | |
| Carbocisteine | 100 | 28 | 18 | 40 | 14 | 86.0 | <0.05 |
| tablet | | | | | | | |

The above results indicate that the three groups of medicines are all effective for treatment of cough, and the tablets of the present invention have better efficacy than those of the control medicines. Moreover, each group had no obvious effect on blood and urine routine test results, liver and kidney functions, thyroid function, blood pressure, heart rate and electrocardiogram, and the incidence of adverse reaction was low. It follows that the medicine of the present invention has a significant efficacy.

### DETAILED EMBODIMENTS

The present invention discloses a compound chemical medicine for respiratory system disease and the preparation process and use thereof. Those skilled in the art can achieve by using the contents herein for reference and improving process parameters appropriately. Specifically, all the similar substitutions and modifications are obvious to those skilled in the art, and they are all deemed to be within the present invention. The product, method and use of the present invention have been described by preferred embodiments. The relevant people could achieve and apply the technique of the present invention by modifying or appropriately changing and combining the method and use described herein without departing from the contents, spirit and scope of the present invention.

In order to better understand the technical solution of the present invention by those skilled in the art, the present invention is further illustrated in detail in combination with detailed examples.

### EXAMPLE 1

60 g Levodropropizine and 250 g carbocisteine were weighed, and prepared into common oral tablets according to the following method:

400 g Starch, 20 g microcrystalline cellulose, 10 g polyvinyl pyrrolidone, 5 g talc and 20 g aspartame were weighed, and the raw materials and the excipients were pulverized and sieved respectively, for later use. Starch was dissolved in water and added with levodropropizine and carbocisteine, grinded into a paste, dried, pulverized, sieved; then fine powders of other excipients were added and mixed evenly, and appropriate amount of gelatin slurry was added, granulated, baked, granule sized, compressed into tablets, to prepare 1000 tablets of the present invention.

### EXAMPLE 2

100 g Levodropropizine and 150 g carbocisteine were weighed, and prepared into an oral liquid according to the following method: levodropropizine and carbocisteine were pulverized and sieved respectively, then mixed evenly. The mixture of them were added with ethanol and stirred, dissolved, then dissolved in about 500 ml purified water, filtered, and the pH was adjusted to 8-9 using 8% dilute ammonia solution. Then appropriate amount of sucrose, sorbic acid and methylparaben were further added, and water was added to 1000 ml, to obtain 1000ml oral liquid of the pharmaceutical composition of the present invention.

### EXAMPLE 3

10 g Levodropropizine and 200 g carbocisteine (appropriate amount) were weighed, and prepared into capsules according to the following method:

50 g Starch, 10 g micronized silica gel, 5 g magnesium stearate, 10 g pregelatinized starch and 10 g low-substituted hydroxymethyl cellulose were weighed. Levodropropizine and carbocisteine were pulverized, sieved respectively, and then mixed evenly. Starch was dissolved in water, and levodropropizine and carbocisteine were added, grinded into a paste, dried, pulverized, and sieved; then fine powders of other excipients were added and mixed evenly, and appropriate amount of gelatin slurry was added, granulated, baked, granule sized, filled into gelatin capsules, to obtain 1000 capsules of the pharmaceutical compositions of the present invention.

### EXAMPLE 4

30 g Levodropropizine and 400 g carbocisteine (appropriate amount) were weighed, and prepared according to the procedure of Example 3, to obtain 1000 capsules of the medicine of the present invention.

### EXAMPLE 5

60 g Levodropropizine and 375 g carbocisteine (appropriate amount) were weighed, and prepared according to the procedure of Example 3, to obtain 1000 capsules of the medicine of the present invention.

### EXAMPLE 6

80 g Levodropropizine and 450 g carbocisteine (appropriate amount) were weighed, and prepared according to the procedure of Example 3, to obtain 1000 capsules of the medicine of the present invention.

### EXAMPLE 7

40 g Levodropropizine and 400 g carbocisteine (appropriate amount) were weighed, and prepared according to the procedure of Example 3, to obtain 1000 capsules of the medicine of the present invention.

### EXAMPLE 8

70 g Levodropropizine and 450 g carbocisteine (appropriate amount) were weighed, and prepared according to the procedure of Example 3, to obtain 1000 capsules of the medicine of the present invention.

A compound chemical medicine for respiratory system disease as well as the preparation process and use thereof proposed by the present invention has been described by examples. Those skilled in the art can achieve the present invention by modifying or appropriately changing and combining the compound chemical medicine for respiratory system disease as well as the preparation process and use thereof described herein, without departing from the contents, spirit and scope of the present invention. Specifically, all the similar substitutions and modifications are obvious to those skilled in the art, and they are all deemed to be within the spirit, scope and contents of the present invention.

1. A pharmaceutical composition for respiratory system disease, wherein the pharmaceutical composition consists of levodropropizine and carbocisteine as active ingredients and a pharmaceutically acceptable excipient.

2. The pharmaceutical composition for respiratory system disease according to claim 1, wherein the active ingredients levodropropizine and carbocisteine are formulated by the weight ratio of 1˜10 parts of levodropropizine to 15˜45 parts of carbocisteine.

3. The pharmaceutical composition for respiratory system disease according to claim 1, wherein the active ingredients levodropropizine and carbocisteine are formulated by the weight ratio of 3˜8 parts of levodropropizine to 20˜40 parts of carbocisteine.

4. The pharmaceutical composition for respiratory system disease according to claim 1, wherein the active ingredients levodropropizine and carbocisteine are formulated by the weight ratio of 4˜7 parts of levodropropizine to 25˜37.5 parts of carbocisteine.

5. The pharmaceutical composition for respiratory system disease according to claim 1, wherein the active ingredients levodropropizine and carbocisteine are formulated by the weight ratio of 6 parts of levodropropizine to 25 parts of carbocisteine.

6. The pharmaceutical composition for respiratory system disease according to claim 1, wherein the active ingredients levodropropizine and carbocisteine are formulated by the weight ratio of 6 parts of levodropropizine to 37.5 parts of carbocisteine.

7. The pharmaceutical composition for respiratory system disease according to claim 1, wherein the pharmaceutical composition is an immediate-release pharmaceutical preparation, a slow-release pharmaceutical preparation or a common pharmaceutical preparation made from the active ingredients and any pharmaceutically acceptable excipient.

8. The pharmaceutical composition for respiratory system disease according to claim 1, wherein the pharmaceutical preparation is an oral preparation.

9. The pharmaceutical composition for respiratory system disease according to claim 8, wherein the oral preparation is a tablet, a capsule, or an oral liquid.
